(19) United States
(12) Patent Application Publication
(10) Pub. No.: US 2013/0019871 A1
(43) Pub. Date: Jan. 24, 2013

(54) Inflatable Oral Airway Apparatus and Method for Using the Same

(71) Applicant: Alexander Nemirovsky, Santa Monica, CA (US)

(72) Inventor: Alexander Nemirovsky, Santa Monica, CA (US)

(21) Appl. No.: 13/189,189

(22) Filed: Jul. 22, 2011

(51) Int. Cl. A61M 16/04

(52) U.S. Cl. 128/207.15

(57) ABSTRACT

An inflatable oral airway (IOA) device for maintaining the patency of an airway of a patient without substantial extension into the pharynx or larynx of the patient. The IOA is inserted into the patient's oro-pharynx after the patient is sedated or under general anesthesia. The IOA comprises a core that is substantially shaped the same as a conventional or normal human airway. The IOA has an inverted orientation, i.e. the bottom or ventral surface of the core rests on the upper surface of the patient's tongue. The IOA also comprises a cuff and a hinge balloon which may be inflated in tandem or in sequence to open and support the soft tissues of the mouth and pharynx in four different directions as well as articulate and increase the internal volume of the IOA itself.

### BACKGROUND

1. Field of the Technology

The disclosure relates to the field of medical devices, specifically upper oral airway devices for maintaining an unobstructed airway path of a patient.

2. Description of the Prior Art

Oral airway tubes have long been used for insertion into the airway of an unconscious patient to provide an unobstructed path for respiration. Many of these oral airway tubes include some kind of inflatable structure or balloon that extends into part of the airway to support or open tissues at the back of the mouth and upper end of the throat. Frequently, these balloons or other articulated structures attempt to provide air sealing in the pharynx.

While these airway tubes are not without their particular successes, significant problems in their use have arisen. An oral airway tube which extends into the open airway (pharynx) or into the upper throat (larynx) requires partial or total anesthetization in order to prevent involuntary choking by the patient. However when a patient is partially or entirely anesthetized, this can cause the soft tissue of the pharynx and larynx to relax and collapse, causing the patient various breathing problems, choking, and other difficulties which may require further intervention on the part of the supervising medical staff.

What is needed is an oral airway tube which extends over the base of the tongue and does not make direct contact with the larynx of the patient in order to prevent laryngospasm, and which can be utilized to open the upper airway in the event of local collapse of surrounding tissue structures by increasing the internal dimensions of the oral airway tube itself.

### BRIEF SUMMARY

An inflatable oral airway device is arranged and configured for insertion into the upper airway of a patient without substantial extension into the pharynx or larynx of the patient. The inflatable oral airway device includes a hollow expandable core with a flat distal end and a mouth piece and teeth guard disposed at its proximal end which defines an open pathway. The expandable core has a predetermined length extending to the back of the mouth but does not substantially extend into the pharynx or larynx of the patient. The core of the device also includes a cuff that is selectively inflatable to extend a supporting surface from the expandable core and is capable of opening and supporting tissue structures in the back of the mouth and adjacent areas of the throat of the patient.

The hollow expandable core of the oral airway device includes a gap defined in its dorsal surface and a hinge defined in its ventral surface. The cuff includes means for expanding into the gap defined in the dorsal surface of the core and increasing its internal volume when it is inflated. A pump for inflating the cuff is communicated to the cuff by a tube.

The invention further provides for an oral airway device arranged and configured for insertion into the upper airway of a patient without making direct contact with the larynx of the patient. The device includes an expandable core defining an open pathway, wherein the expandable core includes at least a dorsal surface and a ventral surface. The expandable core has a predetermined length extending to the back of the mouth but not substantially into the pharynx or larynx of the patient. A hinge balloon is also defined into a surface of the expandable core and is selectively inflatable to articulate and increase the internal volume of the expandable core.

The oral airway device further includes a cuff attached to the expandable core and is selectively inflatable to extend a supporting surface from the expandable core which is capable of opening and supporting tissue structures in the back of the mouth and adjacent areas of the throat.

In one embodiment, the hinge balloon and cuff of the oral airway device are independently inflatable. The oral airway device includes a hinge in its ventral surface and a pump for inflating the hinge balloon communicated by means of a tube.

The invention further provides for a method for maintaining the patency of the airway of a patient without substantial extension into the pharynx or larynx of the patient including defining an open pathway with a hollow expandable core of a inflatable oral airway device, the core having a predetermined length extending to the back of the mouth but not substantially into pharynx or larynx of the patient. At least one portion of the inflatable oral airway device is inflated to expand the hollow core which opens and supports the tissue structures in the back of the mouth and adjacent areas of the throat of the patient, while also increasing the internal volume of the inflatable oral airway device.

In one embodiment, the method further includes inflating a cuff disposed on a dorsal surface of the inflatable oral airway device in order to expand the hollow core. Here, the hinge balloon defined into a surface of the inflatable oral airway device is inflated by manipulating a pump communicated to the hinge balloon by means of a tube.

In another embodiment, the method includes inflating a hinge balloon defined into a surface of the inflatable oral airway device in order to expand the hollow core. The method step of opening and supporting the tissue structures in the back of the mouth and adjacent areas of the throat of the patient includes extending a supporting surface of the cuff from the dorsal surface of the inflatable oral airway device to an area adjacent to the device and expanding its hollow core. Opening and supporting the tissue structures in the back of the mouth and adjacent areas of the throat of the patient may also include extending a supporting surface of the hinge balloon from a surface of the inflatable oral airway device to the area adjacent to the device and expanding its hollow core.

In yet another embodiment, the method step of increasing the internal volume of the inflatable oral airway device includes extending the cuff into a gap defined in the dorsal surface of the inflatable oral airway device in order to expand the hollow core. The internal volume of the inflatable oral airway device is increased by expanding the hinge balloon against at least two opposing surfaces of the device in order to expand its hollow core.

In yet another embodiment, increasing the internal volume of the inflatable oral airway device includes opening a hinge defined in a ventral surface of the inflatable oral airway device in order to expand the hollow core.

In still another embodiment, inflating at least one portion of the inflatable oral airway device also includes inflating a cuff disposed on a dorsal surface of the device and a hinge balloon defined into a surface of the device in tandem in order to expand its hollow core.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The disclosure can be better visualized by turning now to the following drawings wherein like elements are referenced by like numerals.

### BRIEF DESCRIPTION OF THE DRAWINGS

FIG. 1 is a side elevational view of the inflatable oral airway (IOA) device of the current invention. The IOA comprises a cuff which is shown in its deflated configuration.

FIG. 2 is a side elevational view of the IOA device seen in FIG. 1 with the cuff in the inflated configuration.

FIG. 3 is an end elevational view of the IOA device seen in FIG. 1 with the cuff in the deflated configuration.

FIG. 4 is an end elevational view of the IOA device seen in FIG. 2 with the cuff in the inflated configuration.

FIG. 5 is a perpendicular cross sectional view taken through line A-A in FIG. 7 of an alternative embodiment of the IOA.

FIG. 6 is a side elevational view of an alternative embodiment of an IOA device comprising a cuff and hinge balloon in a deflated configuration.

FIG. 7 is a side elevational view of an alternative embodiment of the IOA device seen in FIG. 6 with the cuff and hinge balloon in an inflated configuration.

The disclosure and its various embodiments can now be better understood by turning to the following detailed description of the preferred embodiments which are presented as illustrated examples of the embodiments defined in the claims. It is expressly understood that the embodiments as defined by the claims may be broader than the illustrated embodiments described below.

### DETAILED DESCRIPTION OF THE PREFERRED EMBODIMENTS

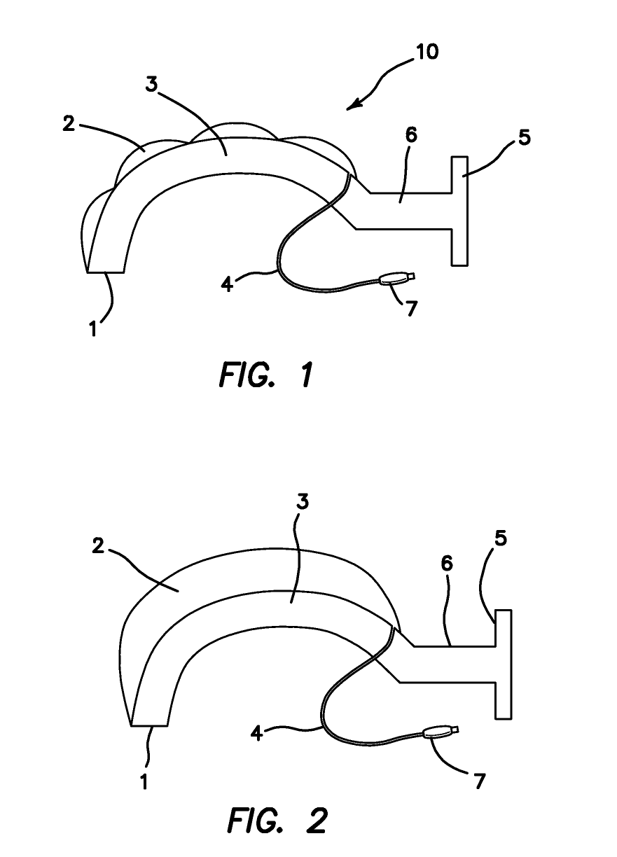

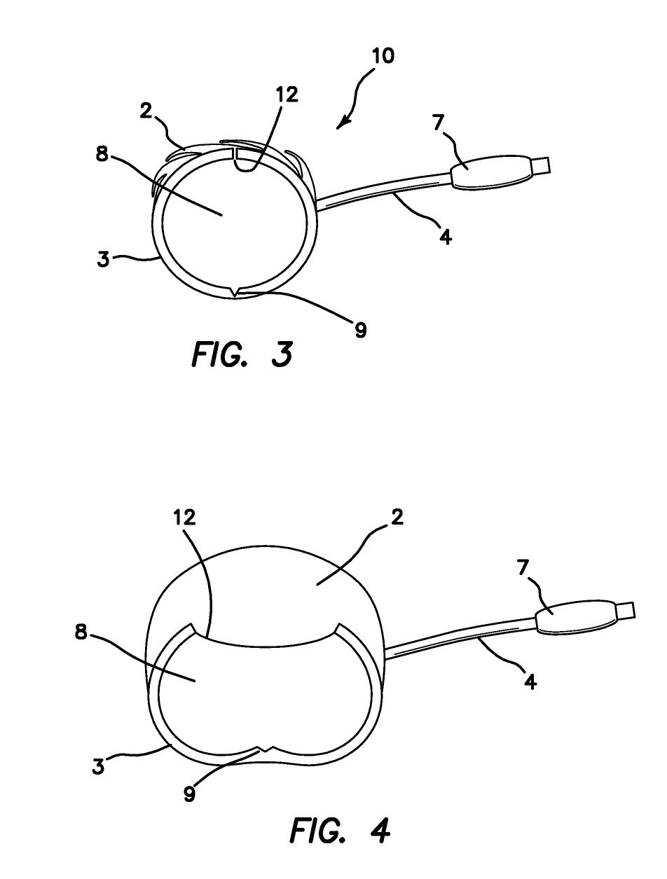

An inflatable oral airway (IOA) device 10 of one the illustrated embodiments of the current invention is shown in its most general form in FIG. 1. The IOA 10 is used initially as many other artificial oral airway devices, namely the IOA 10 is inserted into the patient's oro-pharynx. The IOA 10 is preferably is comprised of plastic or other sanitary material suitable for medical use as is commonly known. The IOA 10 comprises a core 3 that is substantially shaped the same as a conventional or normal human airway. The core 3 comprises a straight mouth piece section 6 with a teeth guard 5 disposed at its most proximal end, and a flat, downwardly directed distal end 1. The IOA 10 has an inverted orientation, i.e. the bottom surface of the core 3 rests on the upper surface of the patient's tongue. The core 3 of the IOA 10 comprises a hollow center or internal channel 8 as best seen in FIGS. 3 and 4 to allow for patency and access to the open airway of the patient. Also as best seen in the frontal view of the IOA 10 shown in FIG. 3, the core 3 comprises a gap 12 defined along its entire upper or dorsal longitudinal length and a hinge 9 defined along its lower or ventral longitudinal length at both the distal end 1 and proximal end of the core 3. In the illustrated embodiment gap 12 and hinge 9 are diametrically opposing each other, but any relative orientation may be employed and where advantage can be realized multiple hinges 9 can be defined into corresponding multiple, selected positions or segments of core 3 to give the need flexibility to core 3 as further discussed below. A second gap (not shown) is created when the hinge 9 is opened. This second gap may further comprise a membrane comprised of a thin plastic disposed across its width which prevents protrusion of the patient's tongue and secretions from getting inside the internal channel 8 and interfering with both ventilation and visibility of the patient's oral airway.

The core 3 of the IOA 10 additionally comprises a cuff 2 coupled to its dorsal surface. Cuff 2 extends across at least a portion of the dorsal surface and across gap 12 to provide a closed inflatable chamber. The cuff 2 is shown in a deflated configuration in FIGS. 1 and 3, and in an inflated configuration in FIGS. 2 and 4. Cuff 2 is pneumatically inflated by a conventional means such as a bulb or syringe 7 communicated to the cuff 2 by a tube 4. When inflated, the cuff 2 near the distal end 1 is utilized to open or support the soft tissue structures at the back of the mouth and upper end of the throat to provide for patency in the event of collapse. It is important to note that the shape of cuff 2 can be varied as needed other than the shape shown in FIGS. 1 and 2 without departing from the original spirit and scope of the invention.

The IOA 10 is first inserted into the mouth of the patient with the cuff 2 deflated. The teeth guard 5 is placed outside patient's teeth to correctly position IOA 10, namely to prevent the IOA 10 from being introduced too far into the patient's pharynx. After the IOA 10 has been correctly inserted as gauged by guard 5, the distal tip 1 of the core 3 will be located above the epiglottis. With the cuff 2 deflated, the IOA 10 establishes an open airway column throughout the core 3 of the device. IOA 10 may be provided in different sizes and/or lengths to accommodate different anatomies of patients, e.g. infants, children, youth and adults.

A user, such as a medical practitioner, then inflates the cuff 2 using manual actuation of the bulb 7 which pumps air into the cuff 2 via the tube 4 through a conventional one-way check valve included as part of bulb 7. The cuff 2 may also be inflated by means of a syringe or other pneumatic device known in the art inserted into the bulb 7. Upon inflation of the cuff 2, the soft tissues of the mouth and pharynx are simultaneously pushed away from the tongue and oral cavity in four different directions, thus increasing the size of the patient's natural airway. At the same time, the inflated cuff 2 expands into the gap 12 defined in the core 3 as seen in FIG. 4, pushing the core 3 apart and thus increasing the diameter of the internal channel 8, thereby increasing the airway volume to or toward the maximum achievable for the specific patient. As the cuff 2 is being inflated, the hinge 9 is actuated and allows the core 3 to continually expand or unfold and accommodate the cuff's 2 increasing volume. Upon complete inflation of the cuff 2, the size of the patient's natural airway is increased with a simultaneous increase in the size internal channel 8 of the IOA 10, thus becoming a superior conduit for the passage of air, oxygen, and other gases down the patient's airway as well as maximizing the accessible oral volume for insertion of endoscopes, instruments and catheters. With the internal channel 8 expanded by the cuff 2, other diagnostic or medical devices such as fiber optic cameras and the like may be inserted into the patient's pharynx and larynx via the internal channel 8 of the IOA 10 without fear of the surrounding soft tissue collapsing and possibly choking the patient.

When the IOA 10 is no longer needed, the cuff 2 is deflated by means known in the art and the IOA 10 is removed from the mouth of the patient. The IOA 10 is then disposed of in the proper manner associated with medical waste.

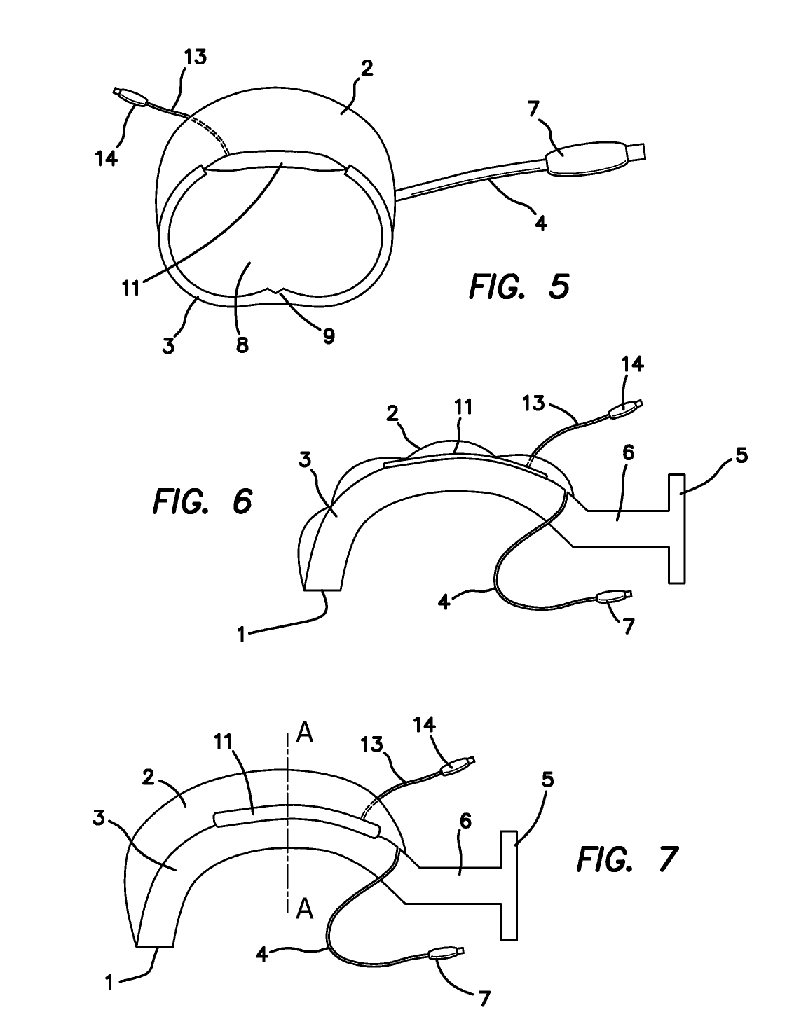

In FIGS. 5-7, an alternative embodiment of an IOA 20 can be seen. Here, the core 3 of the IOA 20 comprises a hinge balloon 11 as its dorsal surface for a portion of its longitudinal length as seen in FIGS. 6 and 7. Communicated to the hinge balloon 11 is a second tube 13 and a corresponding second bulb 14 used to inflate the hinge balloon 11 by means of physical actuation of the bulb 14, or a syringe or other pneumatic device known in the art being inserted into the bulb 14.

In FIGS. 5-7 the hinge balloon 11 is shown as being disposed directly beneath the cuff 2 at the apex of curvature of the core 3, however this is meant to be for illustrative purposes only. The hinge balloon 11 may be defined at any point along the longitudinal length of the core 3 without departing from the original spirit and scope of the invention. Additionally, the hinge balloon 11 may be used to define the lateral or bottom surfaces of the core 3 and may be adjacently disposed to the cuff 2, thus making the IOA 20 articulated at nearly every position or multiple positions if desired. In other words, a multiple number of hinge balloons 11 may be provided in the body of core 3 to provide it an articulated structure that can be selectively unfolded by selectively inflating the multiple hinge balloons 11 depending on their position and articulation with the rigid portions of core 3 with which they are coupled.

The IOA 20 is inserted into the patient's mouth as stated above with both the cuff 2 and hinge balloon 11 deflated as seen in FIG. 6. Once properly inserted, if the patient's requires a larger airway, the medical practitioner may inflate the hinge balloon 11 by means of the bulb 14 and tube 13. As the hinge balloon 11 inflates, it expands against the lateral surfaces of the core 3 as best seen in the cross section of FIG. 5, pushing them apart and flexing the hinge 9 and increasing the volume of internal channel 8. It is in this fashion that the hinge balloon 11 acts as a “hinge muscle” and could be used to open, close, or perform any other kind of articulation of the core 3 desired, depending upon the size and placement of the hinge balloon 11 with respect to the core 3. If the soft tissue surrounding the IOA 20 needs to be pushed back away from the IOA 20, the cuff 2 may then be inflated as described above and as seen in FIGS. 5 and 7, thus clearing the tissue from the airway of the patient and further increasing its overall volume. The cuff 2 and hinge balloon 11 may be inflated simultaneously or in any sequence to differing extents accordingly to the needs of each individual patient. By working the hinge balloon 11 and cuff 2 in tandem, the medical practitioner may achieve expansion of the IOA 20 at a very specific and targeted location within the patient's airway and thus facilitate their specific treatment.

When the IOA 20 is no longer needed, both the cuff 2 and hinge balloon 11 are deflated by means known in the art and the IOA 20 is removed from the mouth of the patient. The IOA 20 is then disposed of in the proper manner associated with medical waste.

In yet another embodiment, the IOA 20 may comprise only a hinge balloon 11 and no cuff 2. In select situations it may not be necessary to push away the soft tissues of the mouth and pharynx and thus the cuff 2 is not needed and does not need to be present on the IOA 20 and increase the overall discomfort of the patient. The hinge balloon 11 alone may be selectively inflated so as to provide means for the IOA 20 to be articulated at nearly every position or multiple positions with a plurality of hinge balloons 11 if desired.

Many alterations and modifications may be made by those having ordinary skill in the art without departing from the spirit and scope of the embodiments. Therefore, it must be understood that the illustrated embodiment has been set forth only for the purposes of example and that it should not be taken as limiting the embodiments as defined by the following embodiments and its various embodiments.

Therefore, it must be understood that the illustrated embodiment has been set forth only for the purposes of example and that it should not be taken as limiting the embodiments as defined by the following claims. For example, notwithstanding the fact that the elements of a claim are set forth below in a certain combination, it must be expressly understood that the embodiments includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the embodiments is explicitly contemplated as within the scope of the embodiments.

The words used in this specification to describe the various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the embodiments.

I claim:

1. An inflatable oral airway device arranged and configured for insertion into the upper airway of a patient without direct contact with the larynx of the patient comprising;
a hollow expandable core with a flat distal end and a mouth piece and teeth guard disposed at its proximal end defining an open pathway, the expandable core having a predetermined length extending to the back of the mouth but not substantially into pharynx or larynx of the patient; and
a cuff coupled to the core, wherein the cuff is selectively inflatable to extend a supporting surface from the expandable core and is capable of opening and supporting tissue structures in the back of the mouth and adjacent areas of the throat of the patient and expanding the hollow core.

2. The oral airway device of claim 1 where the hollow expandable core comprises a gap defined in its dorsal surface and a hinge defined in its ventral surface.

3. The oral airway device of claim 2 where the cuff comprises means for expanding the gap defined in the dorsal surface of the expandable core and increasing the internal volume of the expandable core.

4. The oral airway device of claim 1 further comprising a pump for inflating the cuff communicated to the cuff by means of a tube.

5. An oral airway device arranged and configured for insertion into the upper airway of a patient without direct contact with the larynx of the patient comprising:
an oral airway expandable core defining an open pathway, wherein the expandable core comprises at least a dorsal surface and a ventral surface, the expandable core having a predetermined length extending to the back of the mouth but not substantially into pharynx or larynx of the patient; and
a hinge balloon defined into a surface of the expandable core, wherein the hinge balloon is selectively inflatable to articulate and increase the internal volume of the expandable core.

6. The oral airway device of claim 1 further comprising a cuff coupled to the expandable core, wherein the cuff is selectively inflatable to extend a supporting surface from the expandable core capable of opening and supporting tissue structures in the back of the mouth and adjacent areas of the throat.

7. The oral airway device of claim 6 where the hinge balloon and cuff are independently inflatable.

8. The oral airway device of claim 5 where the expandable core comprises a hinge defined in its ventral surface at both its proximal and distal ends.

9. The oral airway device of claim 5 further comprising a pump for inflating the hinge balloon communicated to the hinge balloon by means of a tube.

10. A method for maintaining the patency of the airway of a patient without making direct contact with the larynx of the patient comprising:
defining an open pathway with a hollow expandable core of a inflatable oral airway device a predetermined length extending to the back of the mouth but not substantially into pharynx or larynx of the patient;
inflating at least one portion of the inflatable oral airway device to expand the hollow core;
opening and supporting the tissue structures in the back of the mouth and adjacent areas of the throat of the patient with the expanded surfaces of the oral airway device; and
increasing the internal volume of the inflatable oral airway device.

11. The method of claim 10 where inflating at least one portion of the inflatable oral airway device comprises inflating a cuff disposed on a dorsal surface of the inflatable oral airway device to expand the hollow core.

12. The method of claim 10 where inflating a portion of the inflatable oral airway device comprises inflating a hinge balloon defined into a surface of the inflatable oral airway device to expand the hollow core.

13. The method of claim 11 where opening and supporting the tissue structures in the back of the mouth and adjacent areas of the throat of the patient comprises extending a supporting surface of the cuff from the dorsal surface of the inflatable oral airway device to the area adjacent to the inflatable oral airway device and expanding the hollow core.

14. The method of claim 11 where increasing the internal volume of the inflatable oral airway device comprises extending the cuff into a gap defined in the dorsal surface of the inflatable oral airway device to expand the hollow core.

15. The method of claim 12 where increasing the internal volume of the inflatable oral airway device comprises expanding the hinge balloon against at least two opposing surfaces of the inflatable oral airway device to expand the hollow core.

16. The method of claim 10 where increasing the internal volume of the inflatable oral airway device comprises opening at least one hinge defined in a ventral surface of the inflatable oral airway device to expand the hollow core.

17. The method of claim 10 where inflating at least one portion of the inflatable oral airway device comprises inflating a cuff disposed on a dorsal surface of the inflatable oral airway device and a hinge balloon defined into a surface of the inflatable oral airway device in tandem to expand the hollow core.

18. The method of claim 11 where inflating the cuff disposed on a dorsal surface of the inflatable oral airway device comprises manipulating a pump communicated to the cuff by means of a tube.

19. The method of claim 12 where inflating the hinge balloon defined into a surface of the inflatable oral airway device comprises manipulating a pump communicated to the hinge balloon by means of a tube.

20. The method of claim 12 where opening and supporting the tissue structures in the back of the mouth and adjacent areas of the throat of the patient comprises extending a supporting surface of the hinge balloon from a surface of the inflatable oral airway device to the area adjacent to the inflatable oral airway device and expanding the hollow core.
